(19) United States
(12) Patent Application Publication
(10) Pub. No.: US 2025/0186009 A1
(43) Pub. Date: Jun. 12, 2025

(54) MEDICAL IMAGE PROCESSING DEVICE, TREATMENT SYSTEM, MEDICAL IMAGE PROCESSING METHOD, AND STORAGE MEDIUM

(71) Applicants: Toshiba Energy Systems & Solutions Corporation, Kawasaki-shi (JP); National Institutes for Quantum Science and Technology, Chiba-shi (JP)

(72) Inventors: Kenta UMENE, Fuchu Tokyo (JP); Yukinobu SAKATA, Kawasaki Kanagawa (JP); Ryusuke HIRAI, Meguro Tokyo (JP); Keiko OKAYA, Setagaya Tokyo (JP); Shinichiro MORI, Chiba-shi (JP)

(73) Assignees: Toshiba Energy Systems & Solutions Corporation, Kawasaki-shi (JP); National Institutes for Quantum Science and Technology, Chiba-shi (JP)

(21) Appl. No.: 19/052,024

(22) Filed: Feb. 12, 2025

Related U.S. Application Data
(63) Continuation of application No. PCT/JP2023/042555, filed on Nov. 28, 2023.

(30) Foreign Application Priority Data
Nov. 29, 2022 (JP) 2022-190393

(51) Int. Cl. A61B 6/00; A61B 6/04; A61B 6/46

(52) U.S. Cl. CPC A61B 6/487; A61B 6/04; A61B 6/461; A61B 6/547

(57) ABSTRACT

According to an embodiment, a medical image processing device includes an image acquirer, a trajectory generator, and a selector. The image acquirer acquires a plurality of fluoroscopic images by imaging a patient. The trajectory generator recognizes a position of a part of interest shown in each of the plurality of fluoroscopic images and generates a trajectory of a state in which the part of interest has moved based on the recognized position of the part of interest. The selector selects a tracking method for tracking the part of interest based on the trajectory of the part of interest.

### CROSS-REFERENCE TO RELATED APPLICATION

This application is based upon and claims the benefit of priority from Japanese Patent Application No. 2022-190393, filed Nov. 29, 2022 and PCT/JP2023/042555, filed Nov. 28, 2023; the entire contents all of which are incorporated herein by reference.

### FIELD

Embodiments of the present invention relate to a medical image processing device, a treatment system, a medical image processing method, and a storage medium.

### BACKGROUND

Radiation treatment is a treatment method for irradiating a tumor (a lesion) within a patient's body with radiation to destroy the tumor. The radiation treatment requires precise targeting of radiation (a treatment beam) with which the tumor is irradiated so that an influence on normal tissues within the patient's body irradiated with the radiation is suppressed. For example, if the tumor is located near the lung or liver, a position of the tumor moves in synchronization with the patient's respiration. For this reason, respiratory-synchronized irradiation in which the treatment beam is radiated in synchronization with the patient's respiration is performed with respect to such tumors. A respiratory-synchronized irradiation method is, for example, a marker tracking method. In the marker tracking method, a marker (a metallic marker) is placed near the tumor inside the patient's body in advance and the marker is tracked by a fluoroscopic image (e.g., an X-ray fluoroscopic image) captured during radiation treatment. In the marker tracking method, control is performed so that the irradiation with the treatment beam is performed only when the marker is projected onto the fluoroscopic image within a predetermined area (irradiation spot) set on the basis of a position of the marker.

Thereby, in the marker tracking method, it is possible to irradiate the tumor with the treatment beam at an appropriate timing and perform the appropriate radiation treatment.

In relation to the marker tracking method, technology for detecting and tracking a metallic marker according to template matching from a fluoroscopic image captured for a period of one or more respiration processes of the patient immediately before treatment has been disclosed (see, for example, Patent Document 1). In the conventional technology, fluoroscopic images are captured in two directions and a position of the marker is three-dimensionally estimated using a triangulation technique. Thereby, in the conventional technology, the position of the marker moving in synchronization with the patient's respiration can be three-dimensionally ascertained and the tumor can be irradiated with the treatment beam at an appropriate timing.

### BRIEF DESCRIPTION OF THE DRAWINGS

FIG. 1 A block diagram showing an example of a configuration of a treatment system including a medical image processing device of a first embodiment.

FIG. 2 A block diagram showing the example of the configuration of the medical image processing device of the first embodiment.

FIG. 3A A diagram showing an example of a template for use in marker recognition in the medical image processing device of the first embodiment.

FIG. 3B A diagram showing an example of a template for use in marker recognition in the medical image processing device of the first embodiment.

FIG. 3C A diagram showing an example of a template for use in marker recognition in the medical image processing device of the first embodiment.

FIG. 3D A diagram showing an example of a template for use in marker recognition in the medical image processing device of the first embodiment.

FIG. 4 A diagram schematically showing an example of a process of recognizing a position of a marker in the medical image processing device of the first embodiment.

FIG. 5A A diagram showing an example of a trajectory of a marker whose position has been recognized in the medical image processing device of the first embodiment.

FIG. 5B A diagram showing an example of a trajectory of a marker whose position has been recognized in the medical image processing device of the first embodiment.

FIG. 6 A flowchart showing a flow of an operation in the medical image processing device of the first embodiment.

FIG. 7 A flowchart showing a flow of an operation for determining whether or not tracking is possible in a selector provided in the medical image processing device of the first embodiment.

FIG. 8 A flowchart showing a flow of another operation for determining whether or not tracking is possible in the selector provided in the medical image processing device of the first embodiment.

FIG. 9 A block diagram showing an example of a configuration of a medical image processing device of a second embodiment.

FIG. 10 A diagram (part 1) showing an example of a display image for presenting information in the medical image processing device of the embodiment.

FIG. 11 A diagram (part 2) showing an example of a display image for presenting information in the medical image processing device of the embodiment.

FIG. 12 A diagram (part 3) showing an example of a display image for presenting information in the medical image processing device of the embodiment.

FIG. 13 A diagram (part 4) showing an example of a display image for presenting information in the medical image processing device of the embodiment.

### DETAILED DESCRIPTION

According to an aspect of the present embodiment, a medical image processing device includes an image acquirer, a trajectory generator, and a selector. The image acquirer acquires a plurality of fluoroscopic images by imaging a patient. The trajectory generator recognizes a position of a part of interest shown in each of the plurality of fluoroscopic images and generates a trajectory of a state in which the part of interest has moved on the basis of the recognized position of the part of interest. The selector selects a tracking method for tracking the part of interest on the basis of the trajectory of the part of interest.

### Advantageous Effects of Invention

Hereinafter, a medical image processing device, a treatment system, a medical image processing method, and a storage medium according to embodiments will be described with reference to the drawings.

### First Embodiment

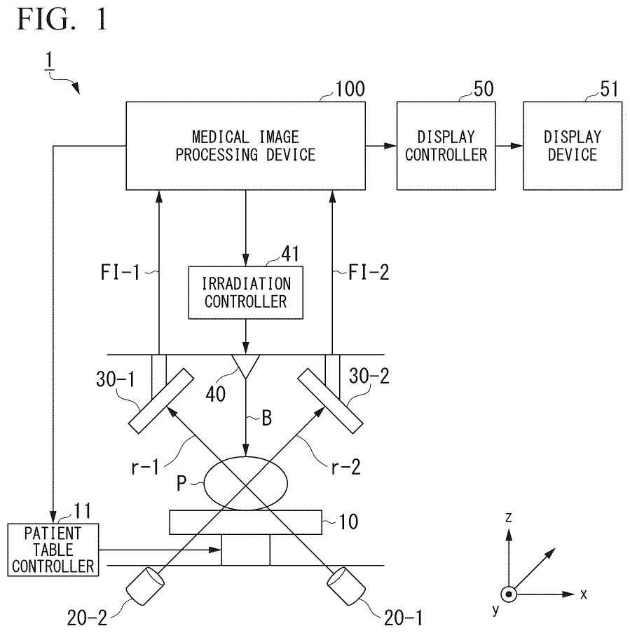

FIG. 1 is a block diagram showing an example of a configuration of a treatment system including a medical image processing device of a first embodiment. A treatment system 1 includes, for example, a treatment table 10, a patient table controller 11, two radiation sources 20 (radiation sources 20-1 and 20-2), two radiation detectors 30 (radiation detectors 30-1 and 30-2), a treatment beam irradiation gate 40, an irradiation controller 41, a display controller 50, a display device 51, and a medical image processing device 100.

Also, a hyphen “-” attached subsequent to a reference numeral shown in FIG. 1 and a number subsequent to the hyphen are used for identifying a corresponding relationship. For example, in the corresponding relationship between the radiation source 20 and the radiation detector 30, a state in which the radiation source 20-1 and the radiation detector 30-1 correspond to each other to form one pair is shown and a state in which the radiation source 20-2 and the radiation detector 30-2 correspond to each other to form another pair is shown. In the following description, when a plurality of identical constituent elements are represented without being distinguished, they are represented without the hyphen “-” and the number subsequent to the hyphen.

The treatment table 10 is a patient table on which a subject (patient) P to be treated with radiation is fixed. The patient table controller 11 controls a translation mechanism and a rotation mechanism provided on the treatment table 10 so that a direction in which the patient P fixed on the treatment table 10 is irradiated with a treatment beam B is changed. The patient table controller 11 can control each of the translation mechanism and the rotation mechanism of the treatment table 10 in three axial directions, i.e., controls the translation mechanism and the rotation mechanism of the treatment table 10 in six axial directions.

The radiation source 20-1 radiates radiation r-1 for seeing through the body of the patient P at a predetermined angle. The radiation source 20-2 radiates radiation r-2 for seeing through the body of the patient P at a predetermined angle different from that of the radiation source 20-1. The radiation r-1 and the radiation r-2 are, for example, X-rays. In FIG. 1, a case in which X-ray photography is performed in two directions on the patient P fixed on the treatment table 10 is shown. Also, the illustration of a controller that controls the irradiation with the radiation r by the radiation source 20 is omitted from FIG. 1.

The radiation detector 30-1 detects the radiation r-1 which has been radiated from the radiation source 20-1 and has arrived at the radiation detector 30-1 after passing through the body of the patient P and generates a two-dimensional X-ray fluoroscopic image FI-1 by imaging a state within the body of the patient P in accordance with a magnitude of energy of the detected radiation r-1. The radiation detector 30-2 detects the radiation r-2 which has been radiated from the radiation source 20-2 and has arrived at the radiation detector 30-2 after passing through the body of the patient P and generates a two-dimensional X-ray fluoroscopic image FI-2 by imaging a state within the body of the patient P in accordance with a magnitude of energy of the detected radiation r-2. The radiation detector 30-1 and the radiation detector 30-2 generate the X-ray fluoroscopic image FI-1 and the X-ray fluoroscopic image FI-2, respectively, at the same timing, i.e., simultaneously. In the radiation detectors 30, the X-ray detectors are arranged in a two-dimensional array shape and generate digital images in which magnitudes of energy of the radiation r arriving at the X-ray detectors are represented by digital values as the X-ray fluoroscopic images FI. The radiation detector 30 is, for example, a flat panel detector (FPD), an image intensifier, or a color image intensifier. In the following description, each radiation detector 30 is assumed to be an FPD. The radiation detector 30 (FPD) outputs the generated X-ray fluoroscopic image FI to the medical image processing device 100. The illustration of a controller that controls the generation of the X-ray fluoroscopic image FI by the radiation detector 30 is omitted from FIG. 1.

A configuration in which the medical image processing device 100 and the radiation detector 30 are connected by a local area network (LAN) or a wide area network (WAN) may be adopted.

In the treatment system 1, the pair of the radiation source 20 and the radiation detector 30 is an example of an “imaging device.” In FIG. 1, an imaging device that captures X-ray fluoroscopic images FI of the patient P in two different directions is shown. In the treatment system 1, the X-ray fluoroscopic image FI is an example of a “fluoroscopic image.” The fluoroscopic image may be, for example, any image, such as a CT image or a DRR image, as long as it is an image onto which a part of interest inside the body of the patient P is projected.

In the treatment system 1, the pair of the radiation source 20 and the radiation detector 30 is configured as one imaging device. In the treatment system 1, because the positions of the radiation source 20 and the radiation detector 30 are fixed, a direction in which the imaging device including the pair of the radiation source 20 and the radiation detector 30 captures images (a relative direction for a fixed coordinate system of the treatment room) is fixed. Therefore, when three-dimensional coordinates are defined in a three-dimensional space in which the treatment system 1 is installed, the positions of the radiation source 20 and the radiation detector 30 can be expressed by coordinate values of three axes. In the following description, information about the coordinate values of the three axes is referred to as geometry information of the imaging device including the pair of the radiation source 20 and the radiation detector 30. Using the geometry information, a position of a tumor (a lesion) inside the body of the patient P, located at any position within predetermined three-dimensional coordinates, can be obtained from the position of the radiation radiated from the radiation source 20 when the radiation passes through the body of the patient P and reaches the radiation detector 30. In other words, the position of the tumor inside the body of the patient P in the predetermined three-dimensional coordinates can be obtained as a projection matrix.

The geometry information can be obtained from installation positions of the radiation source 20 and the radiation detector 30 and inclinations of the radiation source 20 and the radiation detector 30 relative to a reference direction in the treatment room designed when the treatment system 1 is installed. The geometry information can also be obtained from the installation positions of the radiation source 20 and the radiation detector 30 measured by a three-dimensional measurement device or the like. By obtaining a projection matrix from the geometry information, the medical image processing device 100 can calculate a position of the tumor inside the body of the patient P in the three-dimensional space in the captured X-ray fluoroscopic image FI.

In an imaging device capable of simultaneously capturing two X-ray fluoroscopic images FI of the patient P as shown in FIG. 1, a projection matrix can be calculated for each pair of the radiation source 20 and the radiation detector 30. Thereby, it is possible to calculate a coordinate value in predetermined three-dimensional coordinates indicating the position of a part of interest from a position (a two-dimensional coordinate position) of the image of the part of interest captured in the two fluoroscopic images as in the principle of triangulation. The part of interest is a tumor (a lesion), organ, bone, or the like in the body of patient P. The part of interest may be a marker placed in advance near a tumor in the body of patient P. The marker is made of a material, such as a metal, whose image is projected onto the X-ray fluoroscopic image FI by radiation r. Markers may have various shapes, such as a sphere, rod, and wedge. In the following description, the part of interest is assumed to be a marker.

Although the treatment system 1 shown in FIG. 1 has a configuration including two pairs of radiation sources 20 and radiation detectors 30, i.e., two imaging devices, the number of imaging devices provided in the treatment system 1 is not limited to two. For example, the treatment system 1 may include three or more imaging devices (three or more pairs of radiation sources 20 and radiation detectors 30).

The treatment beam irradiation gate 40 radiates radiation for destroying a tumor (a lesion), which is a treatment target site in the patient P's body, as a treatment beam B. The treatment beam B is, for example, a heavy particle beam, X-rays, an electron beam, Y-rays, a proton beam, a neutron beam, or the like. The treatment beam B is linearly radiated to the patient P (e.g., a tumor inside the patient P's body) from the treatment beam irradiation gate 40. The irradiation controller 41 controls the irradiation with the treatment beam B from the treatment beam irradiation gate 40 to the patient P. The irradiation controller 41 causes the treatment beam irradiation gate 40 to radiate the treatment beam B in accordance with a signal indicating an irradiation timing of the treatment beam B output by the medical image processing device 100. In the treatment system 1, the treatment beam irradiation gate 40 is an example of an “irradiator” and the irradiation controller 41 is an example of an “irradiation controller.”

Although the treatment system 1 shown in FIG. 1 has a configuration including one fixed treatment beam irradiation gate 40, the present invention is not limited thereto and the treatment system 1 may have a plurality of treatment beam irradiation gates. For example, the treatment system 1 may further include a treatment beam irradiation gate that irradiates the patient P with a treatment beam in a horizontal direction. The treatment system 1 may be configured so that one treatment beam irradiation gate rotates around the patient P to irradiate the patient P with treatment beams in various directions. For example, the treatment beam irradiation gate 40 shown in FIG. 1 may be configured to be able to rotate 360 degrees around the rotation axis of the horizontal direction Y shown in FIG. 1. The treatment system 1 configured in this way is referred to as a rotating gantry type treatment system. In the rotating gantry type treatment system, the radiation source 20 and the radiation detector 30 also rotate 360 degrees at the same time around an axis identical to the rotation axis of the treatment beam irradiation gate 40. In the treatment system 1 shown in FIG. 1, a configuration in which the pair of the radiation source 20-1 and the radiation detector 30-1 and the pair of the radiation source 20-2 and the radiation detector 30-2 are arranged so that the radiation r-1 and the radiation r-2 intersect each other at angles of +45 degrees formed with respect to the treatment beam B, i.e., the radiation r-1 and the radiation r-2 are perpendicular to each other, when the vertical direction Z in which the treatment beam irradiation gate 40 irradiates the patient P with the treatment beam B is considered to be 0 degrees is shown. However, in the treatment system 1, positions at which the treatment beam irradiation gate 40, the pair of the radiation source 20-1 and the radiation detector 30-1, and the pair of the radiation source 20-2 and the radiation detector 30-2 are arranged are not limited to the example shown in FIG. 1. For example, the treatment system 1 may be configured so that the treatment beam irradiation gate 40 is arranged at a position where the treatment beam B is radiated to the patient P in the vertical direction Z or the horizontal direction Y and may also be configured so that the treatment beam irradiation gate 40 of a position or a configuration where the treatment beam B can be radiated to the patient P from any angle including at least an angle such as 30 degrees, 60 degrees, 120 degrees, or 150 degrees or where an irradiation angle or an irradiation direction of the treatment beam B can be adjusted is arranged. For example, the treatment system 1 may be configured so that a pair of the radiation source 20 and the radiation detector 30 is arranged at a position where beams of radiation r are perpendicular to each other and a pair of the radiation source 20 and the radiation detector 30 is arranged at a position where beams of radiation r form different angles with respect to the treatment beam B. More specifically, the treatment system 1, for example, may be configured so that the pair of the radiation source 20-1 and the radiation detector 30-1 is arranged at a position where the radiation r-1 forms an angle of +30 degrees with respect to the treatment beam B (an angle at which the radiation r-1 is emitted from the 5 o'clock direction and enters in the 11 o'clock direction) and the pair of the radiation source 20-2 and the radiation detector 30-2 is arranged at a position where the radiation r-2 forms an angle of −60 degrees with respect to the treatment beam B (an angle at which the radiation r-2 is emitted in the 8 o'clock direction and enters in the 2 o'clock direction).

The medical image processing device 100, for example, determines whether or not it is possible to track the position of a tumor that moves in synchronization with the respiration of the patient P on the basis of the X-ray fluoroscopic image FI captured in a radiation treatment preparation step, and selects a method for tracking the tumor (a tracking method). The medical image processing device 100 tracks the tumor that moves inside the body of the patient P using the selected tracking method. The medical image processing device 100 tracks the tumor indirectly, for example, by detecting the position of a marker placed in advance inside the body of the patient P. The medical image processing device 100 recognizes an image of a marker inside the body of the patient P (hereinafter referred to as a “marker image”) projected onto an X-ray fluoroscopic image FI captured in the radiation treatment preparation step and tracks the tumor moving inside the body of the patient P on the basis of the position of this marker image in the X-ray fluoroscopic image FI.

A process in which the medical image processing device 100 tracks the tumor is not limited to a method for indirectly detecting a position of a marker (a marker tracking method). A method in which the medical image processing device 100 tracks the tumor may be, for example, a markerless tracking method for indirectly recognizing the position of the tumor on the basis of a shape and movement of an organ near the tumor, i.e., tracking the tumor without using a marker, or may be a method for recognizing and directly tracking the position of the tumor.

The medical image processing device 100 outputs a signal for indicating a predetermined irradiation timing at which the tracked tumor is irradiated with the treatment beam B to the irradiation controller 41.

The medical image processing device 100 outputs information indicating the current state to the display controller 50 so that a state in which the position of the marker or tumor is detected and a state in which the marker or tumor is tracked is presented to a practitioner who performs radiation treatment using the treatment system 1, such as a doctor or technician, i.e., the user of the treatment system 1.

Meanwhile, in the radiation treatment, a treatment plan is made, for example, several days to several weeks in advance. In the treatment planning step, a three-dimensional computed tomography (CT) image is captured and a digitally reconstructed radiograph (DRR) image is generated by virtually reconstructing an X-ray fluoroscopic image FI from the CT image. Also, in the treatment plan, a region of interest (ROI) is decided for tracking the position of the treatment site (tumor) to which the treatment beam B is radiated. In the treatment plan, a direction in which the treatment beam B is radiated to the treatment site (an irradiation direction), the intensity (irradiation intensity) of the radiated treatment beam B, and the like are also decided.

In addition to the process of tracking the tumor and indicating the irradiation timing of the treatment beam B, the medical image processing device 100 performs various types of image processing when the radiation treatment is performed in the treatment system 1. For example, the medical image processing device 100 performs image processing for alignment for aligning the current position of the patient P so that the treatment beam B is radiated in an irradiation direction and an irradiation intensity determined in advance in the treatment planning step or the like. The medical image processing device 100 outputs an image obtained by the image processing, information obtained by the image processing, and the like to the corresponding constituent element. The image processing for alignment of the patient P in the treatment system 1 is the same as in the conventional treatment system. Therefore, a detailed description of an image processing configuration and process in which the medical image processing device 100 aligns the patient P will be omitted.

The display controller 50 causes the display device 51 to display an image for presenting various information in the treatment system 1 to the user including a state in which the tumor inside the body of the patient P has been tracked in the medical image processing device 100. The display controller 50 causes the display device 51 to display, for example, information output by the medical image processing device 100 indicating a state in which the position of the marker or tumor has been detected or a state in which the marker or tumor has been tracked. At this time, the display controller 50 causes the display device 51 to display, for example, various images such as the captured X-ray fluoroscopic images FI or images in which various information is superimposed on these images. The display device 51 is, for example, a display device such as a liquid crystal display (LCD). The user of the treatment system 1 can obtain information for performing radiation treatment using the treatment system 1 by visually confirming the images displayed on the display device 51. The treatment system 1 may be configured to include a user interface such as an operation unit (not shown) operated by the user of the treatment system 1, and to manually operate various functions executed by the treatment system 1.

In the treatment system 1, a configuration in which the medical image processing device 100, the above-described “imaging device” including the pair of the radiation source 20 and the radiation detector 30, the irradiation controller 41, and the display controller 50 are combined may server as a “medical device.” In the treatment system 1, the “medical device” may be configured to include a user interface such as the above-described operation unit (not shown) in addition to the medical image processing device 100, the “imaging device,” the irradiation controller 41, and the display controller 50. In the treatment system 1, the “medical device” may be further configured to be integrated with the display device 51.

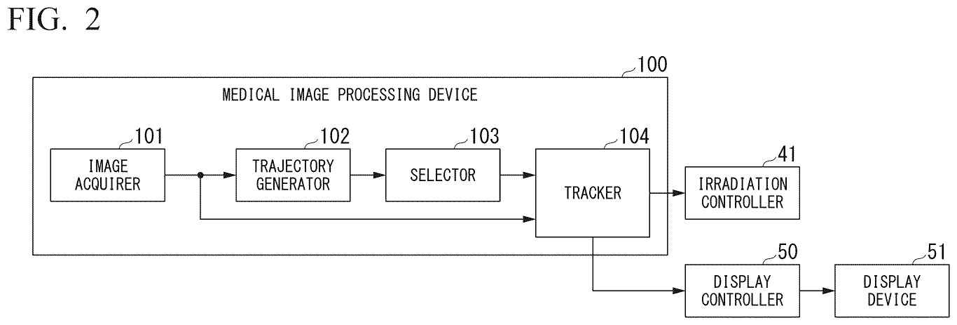

Next, the configuration of the medical image processing device 100 will be described. FIG. 2 is a block diagram showing an example of the configuration of the medical image processing device 100 of the first embodiment. The medical image processing device 100 includes an image acquirer 101, a trajectory generator 102, a selector 103, and a tracker 104. In FIG. 2, the irradiation controller 41 and the display controller 50 (including the display device 51) connected to the medical image processing device 100 are shown together.

Some or all of the constituent elements provided in the medical image processing device 100 are implemented by, for example, a hardware processor such as a central processing unit (CPU) executing a program (software). Also, some or all functions of the above-described constituent elements may be implemented by hardware (including a circuit unit; circuitry) such as a large-scale integration (LSI) circuit, an application-specific integrated circuit (ASIC), a field-programmable gate array (FPGA), or a graphics processing unit (GPU) or may be implemented by software and hardware in cooperation. Also, some or all functions of the above-described constituent elements may be implemented by a dedicated LSI circuit. Here, the program (software) may be stored in advance in a semiconductor memory element such as a read only memory (ROM), a random-access memory (RAM), or a flash memory or a storage device such as a hard disk drive (HDD) (a storage device including a non-transitory storage medium) provided in the medical image processing device 100. The program (software) may be stored in a removable storage medium (a non-transitory storage medium) such as a DVD or a CD-ROM and installed in the storage device provided in the medical image processing device 100 when the storage medium is mounted in a drive device provided in the medical image processing device 100. Also, the program (software) may be downloaded in advance from another computer device via the network and installed in the storage device provided in the medical image processing device 100.

In the radiation treatment preparation step, the image acquirer 101 acquires an X-ray fluoroscopic image FI of the inside of the body of the current patient P fixed on the treatment table 10 in the treatment room to which the treatment system 1 is applied. At this time, the image acquirer 101 continuously acquires a plurality of frames of X-ray fluoroscopic images FI output by the radiation detector 30. In other words, the image acquirer 101 acquires two moving images captured simultaneously in different directions by the imaging device. The image acquirer 101 continuously acquires X-ray fluoroscopic images FI for a period of a length equivalent to one respiratory cycle of the patient P. Furthermore, when the X-ray fluoroscopic images FI of the patient Pis acquired, the image acquirer 101 also acquires geometry information of each X-ray fluoroscopic image FI and associates each acquired X-ray fluoroscopic image FI with the geometry information. The image acquirer 101 may acquire each X-ray fluoroscopic image FI and geometry information from each radiation detector 30 via a LAN or WAN. The image acquirer 101 outputs X-ray fluoroscopic images FI (a plurality of frames of X-ray fluoroscopic images FI) associated with the geometry information to each of the trajectory generator 102 and the tracker 104.

The trajectory generator 102 recognizes a marker image inside the body of the patient P projected within a range designated by the user of the treatment system 1 in each X-ray fluoroscopic image FI output by the image acquirer 101. The range in which the trajectory generator 102 recognizes the marker image, for example, may be a range of the ROI decided in the treatment planning step or may be a predetermined range that includes the marker image in the DRR image generated at the time of treatment planning. The trajectory generator 102 recognizes the marker image within the designated range, for example, according to template matching using a plurality of templates of markers provided in advance. Also, the trajectory generator 102 generates a trajectory of the marker moving in synchronization with the respiration of the patient P on the basis of the position of the marker image recognized in each X-ray fluoroscopic image FI. More specifically, the trajectory generator 102 sequentially connects positions of the marker images recognized in the X-ray fluoroscopic images FI to generate the trajectory of the marker for a period of a length equivalent to at least one respiration cycle of the patient P.

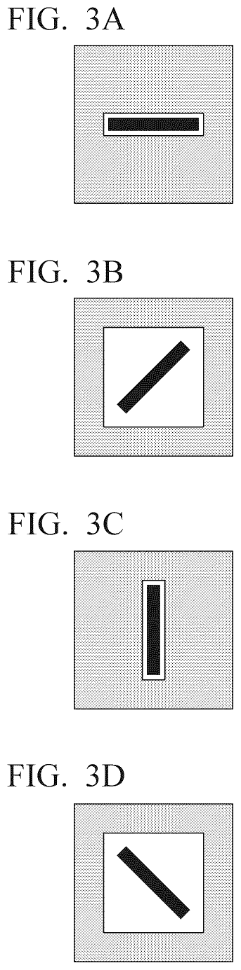

Here, an example of a method for recognizing a marker image in the trajectory generator 102 and generating a trajectory of the marker image will be described. First, an example of a template for recognizing a marker projected onto an X-ray fluoroscopic image FI according to template matching will be described. FIGS. 3A to 3D are diagrams showing an example of a template for use in recognizing a marker in the medical image processing device 100 (more specifically, the trajectory generator 102) of the first embodiment. FIGS. 3A to 3D show examples of a template for recognizing a rod-shaped marker. The template is a two-dimensional image in which a marker image assumed to be projected onto an X-ray fluoroscopic image FI is rotated at a plurality of angles on the image plane. The template shown in FIG. 3A is an example of a template for recognizing a marker projected in the horizontal direction (this angle is set to 0 [deg]) in the X-ray fluoroscopic image FI. The template shown in FIG. 3B is an example of a template for recognizing a marker that is rotated in a right direction and projected (i.e., the right end is projected with an inclination of 45 [deg] in an upward direction) in the X-ray fluoroscopic image FI. The template shown in FIG. 3C is an example of a template for recognizing a marker that is further rotated in the right direction and projected in a vertical direction in the X-ray fluoroscopic image FI (i.e., the right end is projected with an inclination of 90 degrees in the upward direction). The template shown in FIG. 3D is an example of a template for recognizing a marker that is further rotated in the right direction and projected in the X-ray fluoroscopic image FI (i.e., the right end is projected with an inclination of 135 degrees). The templates shown in FIGS. 3A to 3D are merely examples and there are more templates for tracking markers. For example, among templates corresponding to rod-shaped markers, there are templates that recognize markers that are rotated and projected in a depth direction. Likewise, there are a plurality of templates corresponding to markers of different shapes (other than spherical markers) indicating rotation states in various directions.

The trajectory generator 102 recognizes a marker image (according to template matching) by comparing the image projected within a range designated for each of the X-ray fluoroscopic images FI with each of the templates shown in FIGS. 3A to 3D. Also, when a marker is projected onto the X-ray fluoroscopic image FI, the trajectory generator 102 can obtain a position of the marker on an image plane of the X-ray fluoroscopic image FI. In other words, the trajectory generator 102 can obtain the position of the marker in a two-dimensional coordinate system. The position of the marker on the X-ray fluoroscopic image FI can be expressed, for example, by the position of a pixel constituting the X-ray fluoroscopic image FI. The pixel representing the position of the marker on the X-ray fluoroscopic image FI is, for example, a pixel at a center position of the marker image recognized by the trajectory generator 102 on the X-ray fluoroscopic image FI. Here, it is also considered that the entire marker is not projected onto the X-ray fluoroscopic image FI as a marker image, i.e., only a part of the marker is projected as a marker image. In this case, the pixel representing the position of the marker may be a pixel at the center position or a pixel at the end position of a part of the marker image projected onto the X-ray fluoroscopic image FI or may be a pixel corresponding to the center position of the template used when the trajectory generator 102 recognizes the marker image in the X-ray fluoroscopic image FI.

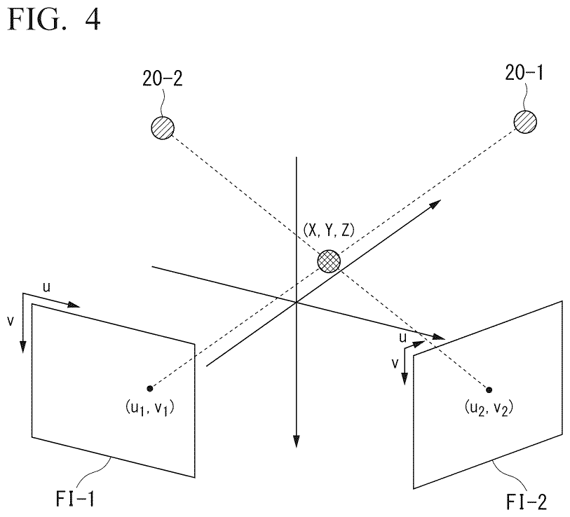

Furthermore, when a marker is projected onto each of two simultaneously captured X-ray fluoroscopic images FI, the trajectory generator 102 can obtain the position of the marker in a three-dimensional coordinate system defined for the treatment room on the basis of the pixel position of the marker and the geometry information associated with the X-ray fluoroscopic images FI. FIG. 4 is a diagram schematically showing an example of a process of recognizing the position of a marker in the medical image processing device 100 (more specifically, the trajectory generator 102) of the first embodiment. In FIG. 4, an example of a case where the position of a marker in three-dimensional coordinates defined for the treatment room is obtained as a projection matrix based on geometry information is schematically shown. In the following description, it is assumed that “→” is located above a reference sign, which denotes coordinates within the three-dimensional coordinates, to express a vector between a position of the coordinates and a position of coordinates corresponding thereto.

In FIG. 4, it is assumed that the trajectory generator 102 recognizes that a marker image is projected onto a position (a pixel position) of coordinates m₁=(u₁, v₁)^{T} on the X-ray fluoroscopic image FI-1 and that a marker image is projected onto a position (a pixel position) of coordinates m₂=(u₂, v₂)^{T} on the X-ray fluoroscopic image FI-2 according to template matching. The trajectory generator 102 calculates a marker position \vec{O}=(X, Y, Z)^{T} in the three-dimensional coordinate system of the treatment room on the basis of a vector \vec{m}₁ between the coordinates m₁ on the X-ray fluoroscopic image FI-1 and the coordinates of the position of the radiation source 20-1 corresponding to the X-ray fluoroscopic image FI-1 and a vector \vec{m}₂ between the coordinates m₂ on the X-ray fluoroscopic image FI-2 and the coordinates of the position of the radiation source 20-2 corresponding to the X-ray fluoroscopic image FI-2, using a relationship of the following Eq. (1) valid for the marker position d.

$$\begin{matrix}[ Math . 1 ] & \\ \begin{matrix}{λ}_{1} [ \begin{matrix}{\vec{m}}_{1} \\ 1\end{matrix} ] = {\vec{P}}_{1} [ \begin{matrix}\vec{O} \\ 1\end{matrix} ] \\ {λ}_{2} [ \begin{matrix}{\vec{m}}_{2} \\ 1\end{matrix} ] = {\vec{P}}_{2} [ \begin{matrix}\vec{O} \\ 1\end{matrix} ]\end{matrix} & ( 1 )\end{matrix}$$

In the above Eq. (1), constants in λ₁, λ₂, \vec{P}₁, and \vec{P}₂ denote fixed-value matrices. Each of λ₁ and λ₂ denotes a (1×3) matrix and each of \vec{P}₁ and \vec{P}₂ denotes a (3×4) projection matrix indicating the three-dimensional coordinates of the treatment room. Here, it is also conceivable that the coordinates m₁ and m₂ of the marker recognized by the trajectory generator 102 according to template matching contain an error in the marker position to some extent. For this reason, the trajectory generator 102 uses least squares based on the relationship in the above Eq. (1) to obtain the position O=(X, Y, Z)^{T} that reduces the error in the three-dimensional position of the marker.

In this way, the trajectory generator 102 obtains a two-dimensional marker position (the coordinates m₁ or the coordinates m₂) on each X-ray fluoroscopic image FI, and performs a calculation process of obtaining a three-dimensional marker position O from the two simultaneously captured X-ray fluoroscopic images FI, for one respiration cycle of the patient P. Also, the trajectory generator 102 sequentially connects two- and three-dimensional marker positions obtained from the continuously captured X-ray fluoroscopic images FI to generate a marker trajectory with a length equivalent to one respiration cycle of the patient P. At this time, it is also conceivable that the trajectory generator 102 does not obtain the two-dimensional marker position in, for example, any one of the frames of the continuously captured X-ray fluoroscopic images FI. In other words, it is also conceivable that the continuity of the two-dimensional marker position or the three-dimensional marker position O is interrupted for a short period of time equivalent to one frame. In this case, the trajectory generator 102 is limited to a short period of time in which the continuity is interrupted, but may supplement the position of the marker to be obtained in the frame in which the position of the marker was not obtained using the position of the marker obtained in the other frame. For example, the trajectory generator 102 may use a position of a marker obtained in a previous frame as a position of a marker to be obtained in the frame where the position of the marker was not obtained or may use an intermediate position between positions of markers obtained in a previous frame and a subsequent frame as a position of a marker to be obtained in a frame where the position of the marker was not obtained.

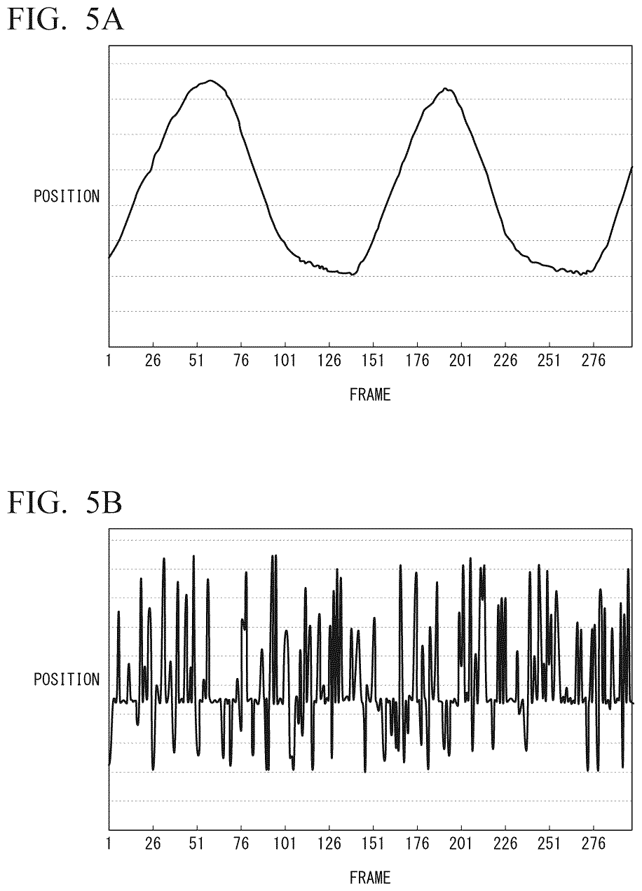

FIGS. 5A and 5B are diagrams showing an example of the trajectory of a marker whose position is recognized by the medical image processing device 100 (more specifically, the trajectory generator 102) of the first embodiment. FIGS. 5A and 5B show examples of a trajectory generated by connecting the positions of the markers recognized in one-direction X-ray fluoroscopic images FI in frame order, i.e., in chronological order. FIGS. 5A and 5B correspond to examples of a trajectory generated by connecting the marker positions in one direction of the three-dimensional position O, i.e., in one direction of the X-axis direction, the Y-axis direction, and the Z-axis direction, in frame order (time series) when the trajectory generator 102 performs a calculation process of obtaining a three-dimensional marker position O from two X-ray fluoroscopic images FI captured at the same time. The trajectory shown in FIG. 5A becomes a trajectory in which the marker position (the pixel position or coordinates) is periodically changed. From this trajectory, it is considered that the patient P is breathing in a stable state. Therefore, it is conceivable that the treatment system 1 can suitably irradiate the tumor (the lesion) with the treatment beam B at a timing based on the marker trajectory. In contrast, the trajectory shown in FIG. 5B becomes a trajectory in which the marker position (the pixel position or coordinates) is changed rapidly. With this trajectory, it is difficult to consider that the treatment system 1 can suitably irradiate the tumor (the lesion) with the treatment beam B. In this case, it may be necessary to wait until the trajectory of the marker position periodically changes like the trajectory shown in FIG. 5A before starting radiation treatment or to switch the method to a method for tracking the tumor without using a marker and suitably radiating the treatment beam B, for example, such as a markerless tracking method.

Returning to FIG. 2, the trajectory generator 102 outputs information indicating the generated trajectory of each marker to the selector 103. More specifically, the trajectory generator 102 outputs information items of information indicating the trajectory of the marker for each template obtained in a two-dimensional coordinate system for each X-ray fluoroscopic image FI (the change in the pixel position) and information indicating the trajectory of the marker for each template obtained in a three-dimensional coordinate system defined in the treatment room for two simultaneously captured X-ray fluoroscopic images FI (the change in the position O) to the selector 103. The information indicating the trajectory of the marker output by the trajectory generator 102 also includes information indicating the template used in template matching. The information indicating the template used in template matching is, for example, identification information exclusively assigned to each template.

The selector 103 determines whether or not a method for tracking the marker in radiation treatment, i.e., a method for indirectly tracking the tumor, can be adopted on the basis of the information indicating the trajectory of each marker output by the trajectory generator 102, and selects a tracking method for tracking the marker or tumor on the basis of a determination result. The selector 103 determines whether or not a method for tracking the marker or tumor can be adopted in radiation treatment, for example, by a classifier using machine learning. In the following description, the process of determining whether or not a method for tracking the marker or tumor can be adopted is referred to as “tracking possibility determination.” The selector 103 performs a tracking possibility determination process for the marker or tumor in radiation treatment, for example, by a classifier using machine learning. As the classifier using machine learning, for example, a classification model such as a random forest, a decision tree, a support vector machine (SVM), a K nearest neighbor algorithm (KNN), or a logistic regression is used. The classification model is a trained model trained in advance using, for example, an artificial intelligence (AI) function, by providing a plurality of trajectories in a two-dimensional coordinate system or a plurality of trajectories in a three-dimensional coordinate system in which it is known whether or not the marker can be tracked. The classifier corresponding to the trajectory in the two-dimensional coordinate system and the classifier corresponding to the trajectory in the three-dimensional coordinate system may use the same classification model or different classification models. Furthermore, in the classifier corresponding to the trajectory in the two-dimensional coordinate system, the classifier corresponding to the trajectory obtained from the X-ray fluoroscopic image FI-1 and the classifier corresponding to the trajectory obtained from the X-ray fluoroscopic image FI-2 may use the same classification model or different classification models.

The selector 103 executes a three-dimensional tracking possibility determination process based on the trajectory of the marker in the three-dimensional coordinate system output by the trajectory generator 102 and a two-dimensional tracking possibility determination process based on the trajectory of the marker in the two-dimensional coordinate system output by the trajectory generator 102. The three-dimensional tracking possibility determination process and the two-dimensional tracking possibility determination process in the selector 103 may be performed at the same time or one of the tracking possibility determination processes may be first performed and the other of the tracking possibility determination processes may be subsequently performed.

When the information about the marker's trajectory obtained in the three-dimensional coordinate system output by the trajectory generator 102 indicates a trajectory that periodically changes in all directions of the three-dimensional position O (i.e., the X-, Y-, and Z-axis directions), for example, as shown in FIG. 5A, the selector 103 selects a tracking method for tracking the marker in the three-dimensional coordinate system (hereinafter referred to as “three-dimensional tracking”). When the information about the marker's trajectory obtained in the three-dimensional coordinate system output by the trajectory generator 102 indicates a trajectory that changes rapidly in any direction of the three-dimensional position O, for example, as shown in FIG. 5B, the selector 103 ascertains the information about the marker's trajectory obtained in the two-dimensional coordinate system output by the trajectory generator 102. Also, when the information about the marker's trajectory obtained in the two-dimensional coordinate system indicates that the trajectory of the X-ray fluoroscopic image FI in one direction is, for example, a trajectory that periodically changes as shown in FIG. 5A, the selector 103 selects a tracking method for tracking the marker in the two-dimensional coordinate system in this X-ray fluoroscopic image FI (hereinafter referred to as “two-dimensional tracking”). When both the information about the trajectory of the marker obtained in the three-dimensional coordinate system and the information about the trajectory of the marker obtained in the two-dimensional coordinate system output by the trajectory generator 102 indicate that the trajectory rapidly changes as shown in FIG. 5B, the selector 103 selects a tracking method that does not use a marker (hereinafter referred to as “external respiratory-synchronized tracking”).

The selector 103 outputs information indicating the selected tracking method to the tracker 104. When the selector 103 selects three-dimensional tracking or two-dimensional tracking, the selector 103 also outputs information about the template used by the trajectory generator 102 according to template matching to the tracker 104.

The tracker 104 tracks the marker image inside the body of the patient P, which are projected within a range designated by the user of the treatment system 1, in each X-ray fluoroscopic image FI output by the image acquirer 101, by the tracking method output by the selector 103. At this time, the tracker 104 tracks the marker image according to template matching using the template indicated in the template information output by the selector 103.

The tracker 104 outputs information indicating a current state in which the marker image has been tracked to the display controller 50. Thereby, the display controller 50 generates a display image for detecting the position of the marker and presenting a current tracking state and causes the display device 51 to display the generated display image, thereby presenting the current state of the treatment system 1 to the user. Furthermore, the tracker 104 generates a signal for indicating an irradiation timing for irradiating the tumor with the treatment beam B on the basis of the tracked marker image and outputs the generated signal to the irradiation controller 41. Thereby, the irradiation controller 41 controls the irradiation with the treatment beam B at the treatment beam irradiation gate 40 so that the treatment beam B is radiated at the irradiation timing output by the tracker 104.

With this configuration, in the medical image processing device 100, the trajectory generator 102 generates a trajectory of the part of interest (a marker in the above-described example) on the basis of the X-ray fluoroscopic image FI captured in the radiation treatment preparation step. Also, in the medical image processing device 100, the selector 103 selects a tracking method for tracking the part of interest on the basis of the trajectory of the part of interest generated by the trajectory generator 102. Furthermore, in the medical image processing device 100, the tracker 104 tracks the part of interest using the tracking method selected by the selector 103 and outputs information for presenting the state in which the part of interest has been tracked to the user and a signal indicating the irradiation timing of the treatment beam B. Thereby, in the treatment system 1 including the medical image processing device 100, it is possible to notify the user whether or not it is possible to track the marker (indirectly track the tumor) using the marker tracking method and it is possible to irradiate the tumor with the treatment beam B at an appropriate timing.

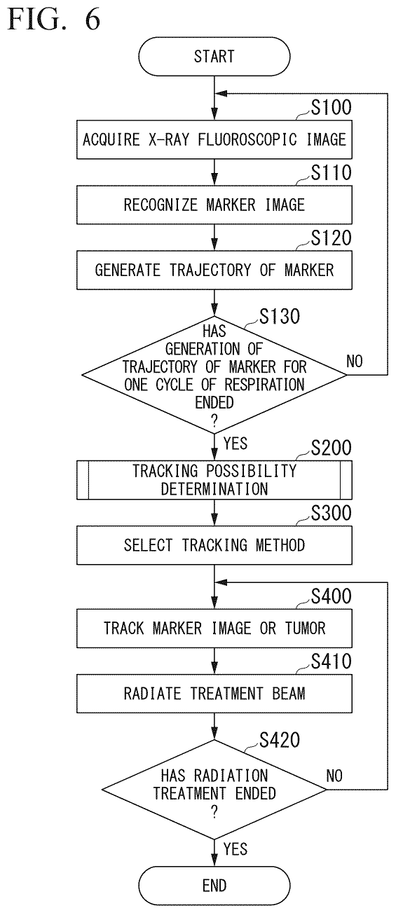

Next, an operation of the medical image processing device 100 will be described. FIG. 6 is a flowchart showing a flow of an operation of the medical image processing device 100 of the first embodiment. In the radiation treatment preparation step of the treatment system 1, the image acquirer 101 acquires each of the first frame X-ray fluoroscopic images FI output by the radiation detector 30 (step S100). The image acquirer 101 associates geometry information corresponding to each acquired X-ray fluoroscopic image FI and outputs an association result to each of the trajectory generator 102 and the tracker 104.

Subsequently, the trajectory generator 102 recognizes a marker image inside the body of the patient P projected within the range designated by the user in each X-ray fluoroscopic image FI of the first frame output by the image acquirer 101 (step S110). Also, the trajectory generator 102 generates a marker trajectory with the position of the marker image recognized in each X-ray fluoroscopic image FI set as the initial position (step S120).

The trajectory generator 102 confirms whether or not the generation of the marker trajectory for one respiratory cycle of the patient P has been completed (step S130). When it is confirmed that the generation of the marker trajectory for one respiratory cycle of the patient P has not ended in step S130, the trajectory generator 102 returns the process to step S100. Thereby, the image acquirer 101 acquires the X-ray fluoroscopic image FI of the next frame in step S100, and the trajectory generator 102 recognizes the marker image projected onto the next frame in step S110 and generates a marker trajectory with the position of the recognized marker image as the next position in step S120. In this way, in the medical image processing device 100, each of the image acquirer 101 and the trajectory generator 102 iterates the process, thereby generating a marker trajectory for one respiratory cycle of the patient P.

On the other hand, when it is confirmed that the generation of the marker trajectory for one respiratory cycle of the patient P has ended in step S130, the trajectory generator 102 outputs information items indicating the positions of the marker images projected onto the X-ray fluoroscopic images FI as a marker trajectory to the selector 103 and moves the process to step S200.

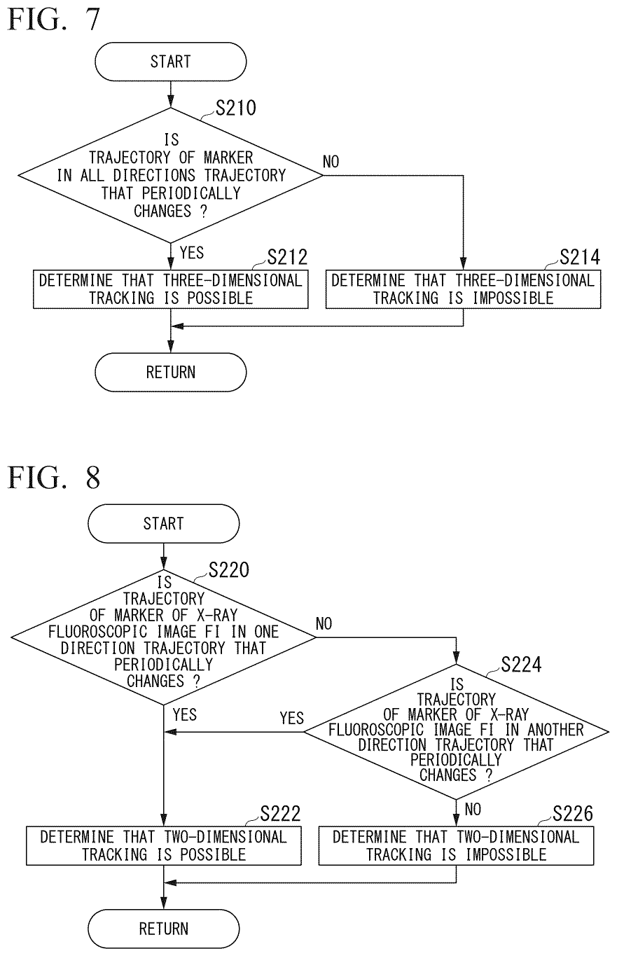

Thereby, the selector 103 executes a tracking possibility determination process of determining whether or not a method for tracking the marker in radiation treatment can be adopted on the basis of information indicating the trajectory of each marker output by the trajectory generator 102 (step S200). Here, the operation of the tracking possibility determination process in the selector 103 will be described in more detail. FIG. 7 is a flowchart showing a flow of the tracking possibility determination operation in the selector 103 provided in the medical image processing device 100 of the first embodiment. In FIG. 7, an example of a flow of an operation of a case where a process of determining whether or not tracking in three dimensions is possible (a process of determining whether or not three-dimensional tracking is possible) is performed on the basis of the marker trajectory in the three-dimensional coordinate system output by the trajectory generator 102 is shown.

In the tracking possibility determination process, the selector 103 uses a corresponding classification model for the information about the marker trajectory obtained in the three-dimensional coordinate system output by the trajectory generator 102 to determine whether or not three-dimensional tracking is possible. First, the selector 103 confirms whether or not the marker trajectory in all directions, i.e., the X-, Y-, and Z-axis directions, at the three-dimensional position O, is a trajectory that periodically changes (step S210). The confirmation in the processing of step S210 is performed for each trajectory generated by the trajectory generator 102, in other words, for each template used by the trajectory generator 102 in template matching to generate the marker trajectory.

When it is confirmed that the trajectory of any one of the markers is a trajectory of the marker that periodically changes in all directions in step S210, the selector 103 determines that the marker can be tracked using the trajectory of the marker in a three-dimensional coordinate system in which all directions change periodically, i.e., that three-dimensional tracking is possible (step S212). Subsequently, the selector 103 returns the process.

On the other hand, when it is confirmed that the trajectory of the marker is a trajectory of the marker that periodically changes in all directions in step S210, the selector 103 determines that the marker is not trackable, i.e., that three-dimensional tracking is impossible, using the trajectory of the marker in the three-dimensional coordinate system (step S214). Subsequently, the selector 103 returns the process.

In this way, the selector 103 performs the three-dimensional tracking possibility determination process. Meanwhile, as described above, the selector 103 executes the three-dimensional tracking possibility determination process based on the trajectory of the marker in the three-dimensional coordinate system output by the trajectory generator 102 and the two-dimensional tracking possibility determination process based on the trajectory of the marker in the two-dimensional coordinate system output by the trajectory generator 102. At this time, in the selector 103, when the three-dimensional tracking possibility determination process and the two-dimensional tracking possibility determination process are performed for the same period, it is only necessary to replace three dimensions in the three-dimensional tracking possibility determination process shown in FIG. 7 with two dimensions for each two-dimensional tracking possibility determination process when the two-dimensional tracking possibility determination process for the X-ray fluoroscopic image FI-1 and the two-dimensional tracking possibility determination process for the X-ray fluoroscopic image FI-2 are performed for the same period.

On the other hand, as described above, the selector 103 may not perform the respective tracking possibility determination processes for the same period. Here, a more detailed operation of a case where the selector 103 does not perform the two-dimensional tracking possibility determination process for the same period will be described. FIG. 8 is a flowchart showing a flow of another tracking possibility determination operation in the selector 103 provided in the medical image processing device 100 of the first embodiment. In FIG. 8, an example of the flow of the operation of a case where the tracking possibility determination processes in two dimensions (the two-dimensional tracking possibility determination processes) for X-ray fluoroscopic images FI are performed sequentially on the basis of the trajectory of the marker in the two-dimensional coordinate system output by the trajectory generator 102 is shown.

In the tracking possibility determination process, the selector 103 uses a corresponding classification model for the information of the trajectory of the marker obtained in the two-dimensional coordinate system for each X-ray fluoroscopic image FI output by the trajectory generator 102 and performs the two-dimensional tracking possibility determination. First, the selector 103 confirms whether or not the marker trajectory based on the two-dimensional pixel position obtained for an X-ray fluoroscopic image FI in one direction (here assumed to be the X-ray fluoroscopic image FI-1) is a trajectory that periodically changes (step S220). The confirmation in the processing of step S220 is performed for each trajectory in the X-ray fluoroscopic image FI-1 generated by the trajectory generator 102, in other words, for each template used in template matching by the trajectory generator 102 to generate the trajectory of the marker projected onto the X-ray fluoroscopic image FI-1.

When it is confirmed that any marker trajectory is a trajectory that periodically changes in step S220, the selector 103 determines that marker tracking can be performed using the trajectory of the marker in the two-dimensional coordinate system that periodically changes in this X-ray fluoroscopic image FI-1, i.e., that two-dimensional tracking is possible (step S222). Subsequently, the selector 103 returns the process.

On the other hand, when it is confirmed that any marker trajectory is not a trajectory that periodically changes in step S220, the selector 103 confirms whether or not the marker trajectory based on the two-dimensional pixel position obtained for the X-ray fluoroscopic image FI in another direction (here, the X-ray fluoroscopic image FI-2) is a trajectory that periodically changes (step S224). This confirmation in the processing of step S224 is also performed for each trajectory in the X-ray fluoroscopic image FI-2 generated by the trajectory generator 102, in other words, for each template used in template matching by the trajectory generator 102 to generate the trajectory of the marker projected onto the X-ray fluoroscopic image FI-2.

When it is confirmed that any marker trajectory is a trajectory that periodically changes in step S224, the selector 103 determines that marker tracking can be performed using the trajectory of the marker in a two-dimensional coordinate system that periodically changes in this X-ray fluoroscopic image FI-2, i.e., that two-dimensional tracking is possible (step S222). Subsequently, the selector 103 returns the process.

On the other hand, when it is confirmed that any marker trajectory is not a trajectory that periodically changes in step S224, the selector 103 determines that marker tracking cannot be performed using the trajectory of the marker in a two-dimensional coordinate system in either the X-ray fluoroscopic image FI-1 or the X-ray fluoroscopic image FI-2, i.e., that two-dimensional tracking is impossible (step S226). Subsequently, the selector 103 returns the process.

In this way, the selector 103 performs the two-dimensional tracking possibility determination process. Meanwhile, as described above, the selector 103 executes the three-dimensional tracking possibility determination process based on the trajectory of the marker in the three-dimensional coordinate system output by the trajectory generator 102 and the two-dimensional tracking possibility determination processes based on the trajectory of the marker in the two-dimensional coordinate system output by the trajectory generator 102. For this reason, the selector 103 may first perform the three-dimensional tracking possibility determination process as shown in FIG. 7 and subsequently perform the two-dimensional tracking possibility determination process as shown in FIG. 8. In contrast, the selector 103 may first perform the two-dimensional tracking possibility determination process as shown in FIG. 8 and subsequently perform the three-dimensional tracking possibility determination process as shown in FIG. 7.

Here, it is considered that the marker can be tracked with higher accuracy in three-dimensional tracking than in two-dimensional tracking in the trajectory of the marker in the treatment system 1. For this reason, when the three-dimensional tracking possibility determination process is first performed and the two-dimensional tracking possibility determination process is subsequently performed, the selector 103 may be configured not to perform the two-dimensional tracking possibility determination process if it is determined that three-dimensional tracking can be performed. That is, the selector 103 may be configured to return the process. On the other hand, when the two-dimensional tracking possibility determination process is first performed and the three-dimensional tracking possibility determination process is subsequently performed, the selector 103 may be configured to perform the three-dimensional tracking possibility determination process even if it is determined that two-dimensional tracking can be performed.

Returning to FIG. 6, after the tracking possibility determination process is executed in step S200, the selector 103 selects a marker tracking method on the basis of a determination result of each of the three-dimensional tracking possibility determination process and the two-dimensional tracking possibility determination process (step S300). In the process of step S300, for example, when a determination result indicates that tracking is possible in both the three-dimensional tracking possibility determination process and the two-dimensional tracking possibility determination process, the selector 103 selects three-dimensional tracking as the marker tracking method. On the other hand, for example, when a determination result indicates that tracking is possible in either the three-dimensional tracking possibility determination process or the two-dimensional tracking possibility determination process, the selector 103 selects a tracking method in which tracking is possible in the determination result as a marker tracking method. Also, for example, when the determination result indicates that tracking is impossible in both the three-dimensional tracking possibility determination process and the two-dimensional tracking possibility determination process, the selector 103 selects a markerless tracking method (external respiratory-synchronized tracking) in which tracking is performed by indirectly or directly determining the position of the tumor without using a marker as a tracking method. The selector 103 outputs information indicating the selected tracking method to the tracker 104.

Thereby, the tracker 104 tracks a marker image or a tumor projected onto each X-ray fluoroscopic image FI output by the image acquirer 101 in the tracking method output by the selector 103 (step S400). Also, when radiation treatment is started, the tracker 104 outputs a signal for indicating an irradiation timing for irradiating the tumor with the treatment beam B to the irradiation controller 41 and causes the treatment beam irradiation gate 40 to radiate the treatment beam B (step S410). Subsequently, the tracker 104 iterates a process of tracking the marker image or the tumor in the processing of step S400 and controlling the irradiation with the treatment beam B in step S410 until the radiation treatment ends (until it is confirmed that the radiation treatment ends in step S420). The process and operation (control) in the radiation treatment step in the treatment system 1 are similar to those in the conventional treatment system. Therefore, a detailed description of the process and operation (control) performed by the medical image processing device 100 in the radiation treatment step will be omitted.

According to this process, in the medical image processing device 100, the trajectory generator 102 generates a trajectory of a part of interest (the marker in the above-described example) on the basis of the X-ray fluoroscopic image FI captured in the radiation treatment preparation step. Also, in the medical image processing device 100, the selector 103 selects a tracking method for tracking the part of interest by performing part-of-interest tracking possibility determination for each template used in template matching for generating the trajectory of the part of interest on the basis of the trajectory of the part of interest generated by the trajectory generator 102. Subsequently, in the medical image processing device 100, the tracker 104 tracks the part of interest using the tracking method selected by the selector 103 and controls the irradiation with the treatment beam B when radiation treatment is started.

Although the configuration in which the trajectory generator 102 recognizes the part of interest projected onto the X-ray fluoroscopic image FI and generates a trajectory for tracking the part of interest has been shown in the medical image processing device 100, the part of interest recognized to generate a trajectory is not limited to the part of interest projected onto the X-ray fluoroscopic image FI. For example, the trajectory generator 102 may generate the trajectory of the part of interest on the basis of any image, such as a CT image or a DRR image, as long as it is an image in which the part of interest inside the body of the patient P can be recognized. Furthermore, although a configuration in which the trajectory generator 102 recognizes the part of interest according to template matching is shown in the medical image processing device 100, the part of interest may be recognized by a method other than template matching. For example, the trajectory generator 102 may recognize the part of interest by detecting a contour using a gradient of luminance in the part of interest in the X-ray fluoroscopic image FI or DRR image as a feature quantity or may recognize the part of interest using a deep learning model in which features of the part of interest have been learned using deep learning, which is a type of machine learning.

Although a configuration in which the trajectory of the part of interest generated by the trajectory generator 102 is input to a classifier to perform a process of determining whether or not the marker or tumor can be tracked is shown in the medical image processing device 100, the tracking possibility determination process of the classifier is not limited to a process to be performed on the basis of the trajectory of the part of interest. For example, the trajectory generator 102 outputs a position of the part of interest (a pixel position) obtained in the two-dimensional coordinate system or the position of the part of interest obtained in the three-dimensional coordinate system to the selector 103 as it is without generating a trajectory of the part of interest and the selector 103 inputs the displacement of the position of the part of interest in each of the two-dimensional coordinate system and the three-dimensional coordinate system (a position difference between two consecutive frames) to the classifier, such that the tracking possibility determination process for the marker or tumor may be performed. In this case, it is only necessary for the process and operation (control) of the trajectory generator 102 or the selector 103 and the process and operation (control) of each of the constituent elements provided in the medical image processing device 100 to be equivalent to the process and operation (control) in the first embodiment described above.

Although the trajectory generator 102 and the selector 103 end their respective processes and operations (control) after the selector 103 selects the tracking method for the part of interest in the medical image processing device 100, they may be configured to continue their processes and operations (control). In this case, even if tracking of the part of interest using the selected tracking method becomes difficult during radiation treatment, it becomes possible to switch the method to a different tracking method more quickly.

### Second Embodiment

A second embodiment will be described below. A configuration of a treatment system including a medical image processing device of the second embodiment is a configuration in which the medical image processing device 100 in the configuration of the treatment system 1 including the medical image processing device 100 of the first embodiment shown in FIG. 1 is replaced with the medical image processing device of the second embodiment (hereinafter referred to as a “medical image processing device 200”). In the following description, the treatment system including the medical image processing device 200 is referred to as a “treatment system 2.”

In the following description, the constituent elements of the treatment system 2 including the medical image processing device 200 similar to those of the treatment system 1 including the medical image processing device 100 of the first embodiment are denoted by the same reference signs and a detailed description of the constituent elements will be omitted. In the following description, only a configuration, operation, and process of the medical image processing device 200, which is a constituent element different from the medical image processing device 100 of the first embodiment, will be described.

Like the medical image processing device 100 of the first embodiment, the medical image processing device 200 determines whether or not it is possible to track the position of a tumor moving in synchronization with the respiration of a patient P, for example, on the basis of an X-ray fluoroscopic image FI captured in a radiation treatment preparation step, and selects a method for tracking the tumor (a tracking method). Like the medical image processing device 100, the medical image processing device 200 also tracks the tumor moving inside the body of the patient P using the selected tracking method and controls a process of irradiating the tracked tumor with a treatment beam B.

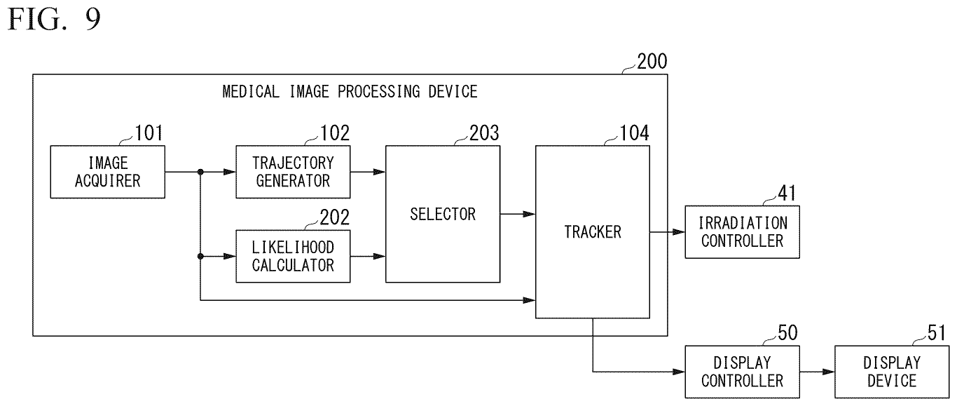

The configuration of the medical image processing device 200 will be described below. FIG. 9 is a block diagram showing an example of the configuration of the medical image processing device 200 of the second embodiment. The medical image processing device 200 includes an image acquirer 101, a trajectory generator 102, a likelihood calculator 202, a selector 203, and a tracker 104. In FIG. 9, an irradiation controller 41 and a display controller 50 (including a display device 51) connected to the medical image processing device 200 are also shown.

The medical image processing device 200 has a configuration in which a likelihood calculator 202 is added to the medical image processing device 100, and accordingly, the selector 103 is replaced with the selector 203. The other constituent elements of the medical image processing device 200 are similar to those of the medical image processing device 100. Therefore, in the following description, the constituent elements of the medical image processing device 200 similar to those of the medical image processing device 100 are denoted by the same reference signs and a detailed description of the respective constituent elements will be omitted. In the following description, only constituent elements different from those of the medical image processing device 100 will be described.

Like the trajectory generator 102 provided in the medical image processing device 100, the likelihood calculator 202 recognizes a marker image inside the body of the patient P projected within a range designated by the user of the treatment system 1 in each X-ray fluoroscopic image FI output by the image acquirer 101. The likelihood calculator 202 may be configured to acquire a result of recognizing the marker image from the trajectory generator 102. In this case, the likelihood calculator 202 can reduce a processing load required for recognizing the marker image. The likelihood calculator 202 calculates the likelihood of the recognized marker image for each X-ray fluoroscopic image FI. Here, the likelihood of the marker image calculated by the likelihood calculator 202 is a value indicating the certainty (similarity) of the marker image within the designated range. The likelihood calculator 202 calculates a likelihood between the marker of the template for use in template matching and the marker projected onto the X-ray fluoroscopic image FI for each X-ray fluoroscopic image FI. The likelihood has a larger value when the similarity to the template marker is higher (the likelihood has a largest value when the image is the template marker image). The likelihood has a smaller value when the similarity to the template marker is lower.

In the configuration of the medical image processing device 200 shown in FIG. 9, the likelihood calculator 202 is provided in parallel with the trajectory generator 102. Therefore, the likelihood calculator 202 calculates a likelihood for the X-ray fluoroscopic image FI of each frame for which the trajectory generator 102 generates a trajectory for the same period during which the trajectory generator 102 generates the trajectory of the marker image. The present invention is not limited to a process in which the calculation of the likelihood in the likelihood calculator 202 and the generation of the trajectory of the marker image in the trajectory generator 102 are performed for the same period. For example, the likelihood calculator 202 may first calculate the likelihood and the trajectory generator 102 may subsequently generate the trajectory of the marker image. In this case, the trajectory generator 102 may reduce a processing load required for generating the trajectory of the marker image if the trajectory of the marker image is generated using a predetermined number of templates starting from the highest likelihood.

The likelihood calculator 202 outputs information indicating the likelihood of the marker image calculated for the X-ray fluoroscopic image FI of each frame to the selector 203. The likelihood calculator 202 may designate a plurality of frames for a period of a length equivalent to one respiratory cycle of the patient P as one unit, calculate an average value (an average likelihood) of likelihoods of marker images calculated for the X-ray fluoroscopic images FI of all frames included in this unit, and output information indicating the average likelihood to the selector 203.

The selector 203 selects a tracking method for tracking a marker or a tumor in radiation treatment on the basis of information indicating the trajectory of each marker output by the trajectory generator 102 and information indicating the likelihood of the marker image projected onto the X-ray fluoroscopic image FI output by the likelihood calculator 202. Although the selection of the tracking method in the selector 203 is similar to that of the selector 103 provided in the medical image processing device 100, information indicating the likelihood of the marker image is also input to the selector 203. Therefore, the selector 203 can select a tracking method for tracking a marker or a tumor from an X-ray fluoroscopic image FI in which a likelihood of the marker image is the highest, i.e., to which a template optimal for radiation treatment corresponds. The selector 203 may be configured to select a tracking method for tracking a marker or a tumor from the X-ray fluoroscopic images FI to which a predetermined number of templates starting from the highest likelihood correspond. In these cases, for the tracking method selection process of the selector 203, the likelihood of the marker image is only considered in the tracking method selection process of the selector 103 described with reference to FIGS. 6 to 8. The tracking method selection process of the selector 203 can be easily considered on the basis of the tracking method selection process in the selector 103. Therefore, a detailed description of the tracking method selection process of the selector 203 will be omitted.

Like the selector 103, the selector 203 outputs information indicating the selected tracking method to the tracker 104. Like the selector 103, when three-dimensional tracking or two-dimensional tracking is selected, the selector 203 also outputs information about the template used by the trajectory generator 102 for template matching (the template with the highest likelihood of the marker image or a predetermined number of templates starting from the highest likelihood) to the tracker 104.

With such a configuration and process, in the medical image processing device 200, as in the medical image processing device 100 of the first embodiment, the trajectory generator 102 generates a trajectory of the part of interest (the marker in the above-described example) on the basis of the X-ray fluoroscopic image FI captured in the radiation treatment preparation step. Furthermore, in the medical image processing device 200, the likelihood calculator 202 calculates a likelihood of the marker image for each X-ray fluoroscopic image FI. Also, in the medical image processing device 200, the selector 203 selects a tracking method for tracking the part of interest on the basis of the trajectory of the part of interest generated by the trajectory generator 102 and the likelihood of the marker image calculated by the likelihood calculator 202. Furthermore, even in the medical image processing device 200, as in the medical image processing device 100, the tracker 104 tracks the part of interest using the tracking method selected by the selector 103 and outputs information for presenting a state in which the part of interest has been tracked to the user and a signal indicating the irradiation timing of the treatment beam B. Thereby, in the treatment system 2 including the medical image processing device 200, as in the treatment system 1 including the medical image processing device 100, it is possible to notify the user whether or not it is possible to track the marker (indirectly track the tumor) in the marker tracking method and it is possible to irradiate the tumor with the treatment beam B at an appropriate timing.

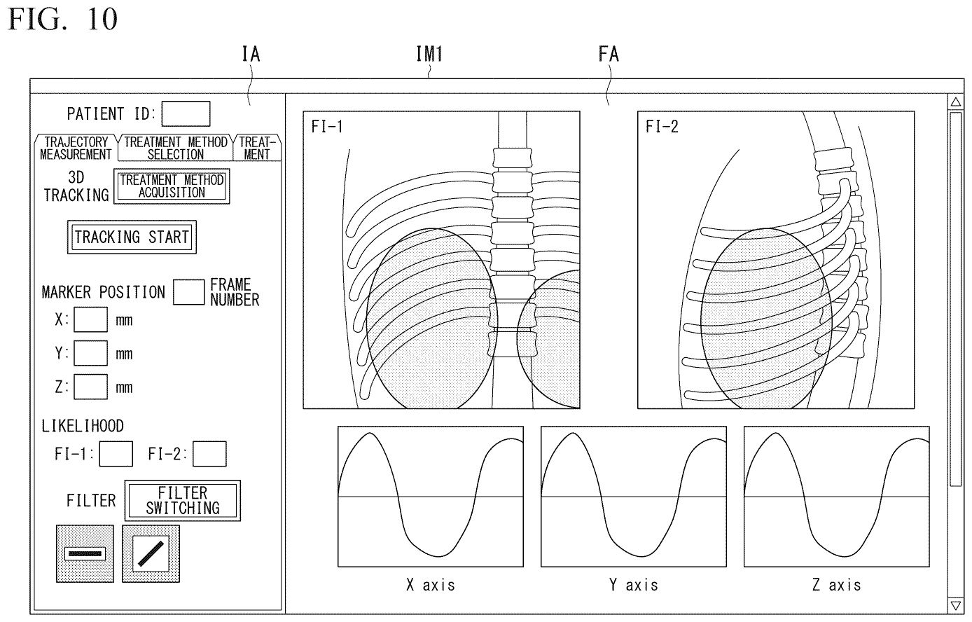

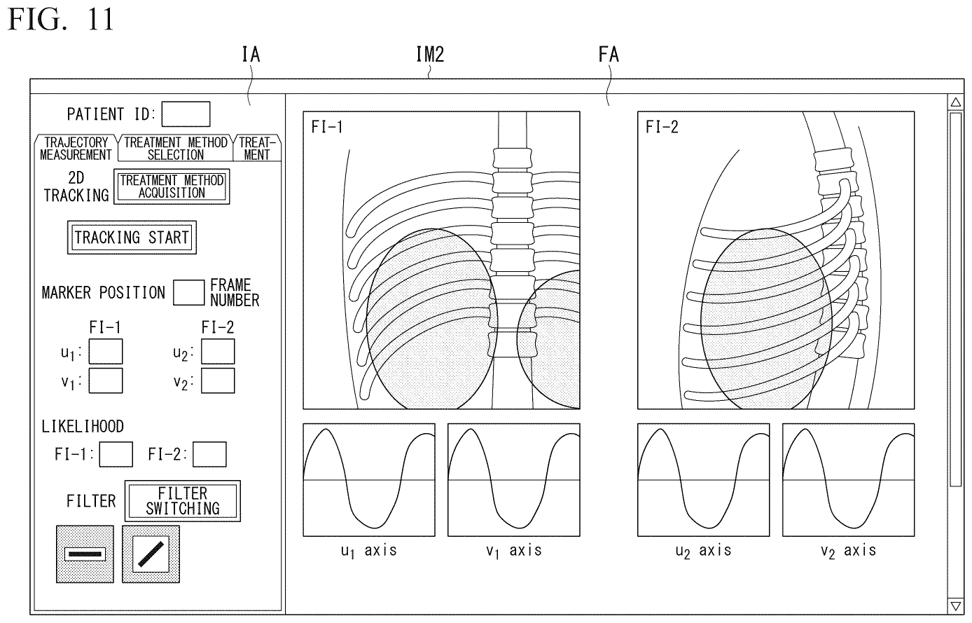

Here, an example of information presented to the user as to whether or not it is possible to track the marker in the medical image processing device 100 or the medical image processing device 200 will be described. In the following description, it is assumed that the medical image processing device 200 presents the information to the user. FIGS. 10 and 11 are diagrams showing an example of a display image for presenting information in the medical image processing device of the embodiment (here, the medical image processing device 200).

In FIG. 10, an example of a graphical user interface (GUI) image IM1 showing a state in which the position of the marker has been detected in a three-dimensional coordinate system is shown. In the GUI image IM1, the X-ray fluoroscopic images FI-1 and FI-2 in which the marker has been detected and the trajectory of the marker in each of the directions (X-, Y-, and Z-axis directions) calculated in a three-dimensional coordinate system are displayed in a line graph in a fluoroscopic image display area FA. Furthermore, in the GUI image IM1, information about the detected marker is displayed in an information display area IA. More specifically, in the GUI image IM1, information indicating that a marker for three-dimensional tracking has been detected, information indicating the three-dimensional position O of the currently detected marker, information indicating a likelihood of the currently detected marker image, information about a template used for detection, and the like are displayed in the information display area IA.

In FIG. 11, an example of a GUI image IM2 indicating a state in which the position of the marker has been detected in a two-dimensional coordinate system is shown. In the GUI image IM2, X-ray fluoroscopic images FI-1 and FI-2 in which a marker is detected are presented in a fluoroscopic image display area FA and a trajectory of the marker in respective directions (u- and v-axis directions) indicating a pixel position obtained in the two-dimensional coordinate system on the X-ray fluoroscopic image FI is presented in a line graph in association with each X-ray fluoroscopic image FI. More specifically, a trajectory of the marker in u₁- and v₁-axis directions indicating the pixel position is presented in a line graph in association with the X-ray fluoroscopic image FI-1 and a trajectory of the marker in u₂- and v₂-axis directions indicating the pixel position is presented in a line graph in association with the X-ray fluoroscopic image FI-2. Furthermore, in the GUI image IM2, information about the detected marker is presented in an information display area IA. More specifically, in the GUI image IM2, information indicating that a marker for two-dimensional tracking has been detected, information indicating the two-dimensional pixel position of the marker currently detected on each X-ray fluoroscopic image FI, information indicating a likelihood of the currently detected marker image, information indicating a template used for detection, and the like are presented in the information display area IA.

The user can confirm the information presented by the GUI image IM1 or GUI image IM2 and control the tracking of the marker by operating a user interface such as an operation unit (not shown). More specifically, the user can switch the marker tracking method or the template used for tracking by performing a “tracking method switching” operation or a “filter switching” operation in the information display area IA, and can start tracking of the marker according to a “tracking start” operation in the information display area IA.

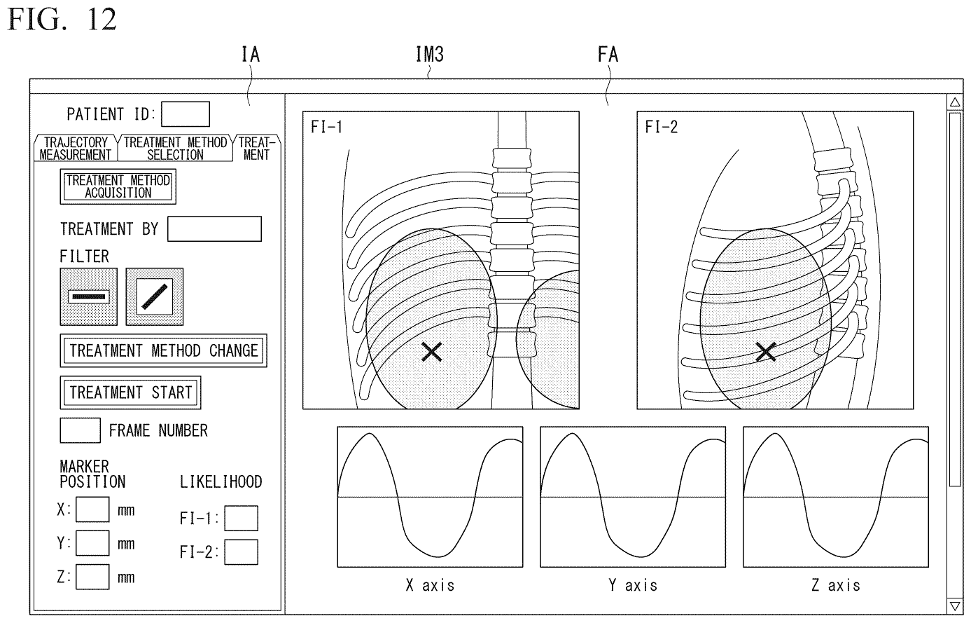

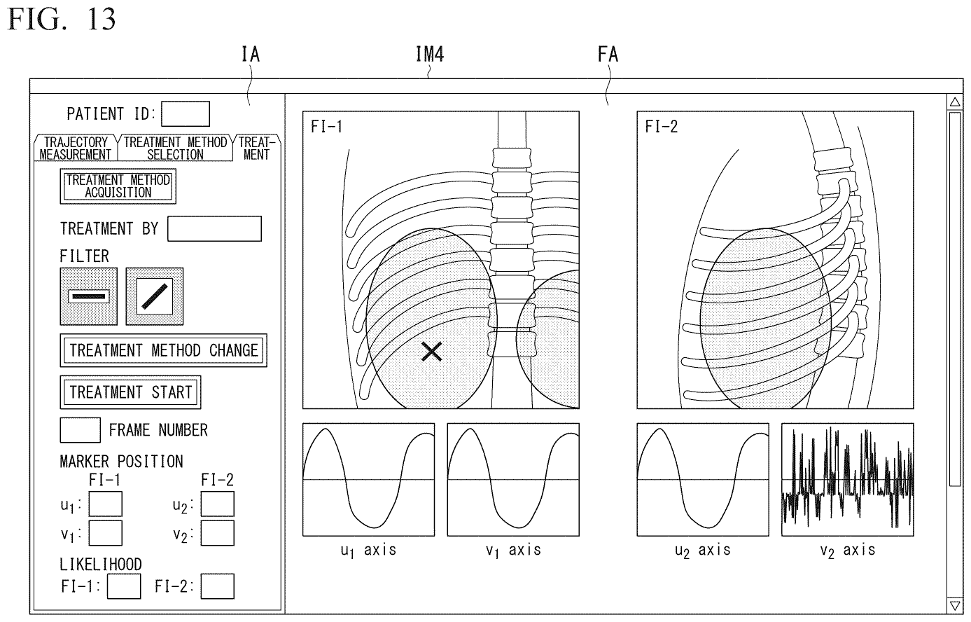

FIGS. 12 and 13 are diagrams showing another example of a display image for presenting information in the medical image processing device of the embodiment (here, the medical image processing device 200).

In FIG. 12, an example of a GUI image IM3 showing a state in which a marker has been three-dimensionally tracked is shown. In the GUI image IM3, X-ray fluoroscopic images FI-1 and FI-2 in which the marker has been tracked and a trajectory of the marker in respective directions (X-, Y-, and Z-axis directions) calculated in a three-dimensional coordinate system are presented in a line graph in a fluoroscopic image display area FA. In the GUI image IM3, a “mark X” indicating a position of the tracked marker is also presented in each of the X-ray fluoroscopic images FI-1 and FI-2. The mark indicating the position of the tracked marker is not limited to the “mark X” and may be a “mark O,” any mark with a different color, or the like as long as the position of the marker can be highlighted. Furthermore, in the GUI image IM3, information about the tracked marker is presented in the information display area IA. More specifically, in the GUI image IM3, information such as information indicating the template used for tracking, information indicating a three-dimensional position O of the marker being three-dimensionally tracked, and information indicating a likelihood of the marker image is presented in the information display area IA.

In FIG. 13, an example of a GUI image IM4 showing a state in which a marker has been two-dimensionally tracked is shown. In the GUI image IM4, X-ray fluoroscopic images FI-1 and FI-2 in which the marker has been tracked are presented in a fluoroscopic image display area FA and a trajectory of the marker in respective directions (u- and v-axis directions) indicating the position of the pixel obtained in the two-dimensional coordinate system on the X-ray fluoroscopic image FI is presented as a line graph in association with each X-ray fluoroscopic image FI. More specifically, the trajectory of the marker in the u₁- and v₁-axis directions indicating the pixel positions is presented in a line graph in association with the X-ray fluoroscopic image FI-1 and the trajectory of the marker in the u₂- and v₂-axis directions indicating the pixel positions is presented in a line graph in association with the X-ray fluoroscopic image FI-2. In the GUI image IM4, a “mark X” indicating the position of the tracked marker only in the X-ray fluoroscopic image FI-1 is presented. This is because in the X-ray fluoroscopic image FI-2, the trajectory of the pixel position in the v₂-axis direction of the tracked marker image is a trajectory that changes rapidly, so that the position of the marker cannot be tracked. Furthermore, in the GUI image IM4, information about the tracked marker is presented in the information display area IA. More specifically, in the GUI image IM4, information indicating a template used for tracking, information indicating a two-dimensional pixel position of the marker being two-dimensionally tracked, information indicating a likelihood of the marker image, and the like are presented in the information display area IA.

The user can confirm the information presented by the GUI image IM3 or the GUI image IM4 and control a process of irradiating the tumor with the treatment beam B by operating a user interface such as an operation unit (not shown). More specifically, the user can confirm content of the radiation treatment to be currently performed according to a “treatment method change” operation or a “treatment method acquisition” operation in the information display area IA and can start the process of irradiating the tumor with the treatment beam B by performing a “treatment start” operation in the information display area IA.

As described above, in the medical image processing device of each embodiment, the trajectory generator generates a trajectory of a part of interest (e.g., a marker) on the basis of a two-dimensional fluoroscopic image (e.g., an X-ray fluoroscopic image) captured in the radiation treatment preparation step. Also, in the medical image processing device of each embodiment, the selector selects a tracking method for tracking the part of interest. In other words, in the medical image processing device of each embodiment, the selector automatically determines whether or not the part of interest is trackable and selects a template to be used for tracking. Subsequently, the medical image processing device of each embodiment tracks the part of interest using the selected tracking method. Thereby, the treatment system including the medical image processing device of each embodiment can determine whether or not radiation (a treatment beam) can be radiated in synchronization with the respiration of the subject (a patient) on the basis of the fluoroscopic image (respiratory-synchronized irradiation). Furthermore, the treatment system including the medical image processing device of each embodiment can present a state in which the position of the part of interest has been detected and a state in which the part of interest has been tracked to the user and can irradiate a tumor (a lesion) in the body of the subject with radiation at a suitable timing.

According to at least one embodiment described above, there are provided an image acquirer (101) configured to acquire a plurality of fluoroscopic images (e.g., X-ray fluoroscopic images FI) by imaging a patient (P); a trajectory generator (102) configured to recognize a position of a part of interest (e.g., a marker) shown in each of the plurality of fluoroscopic images and generate a trajectory of a state in which the part of interest has moved on the basis of the recognized position of the part of interest; and a selector (103) configured to select a tracking method for tracking the part of interest when treatment is performed for a patient on the basis of the trajectory of the part of interest, whereby it is possible to determine whether or not respiratory-synchronized irradiation with the treatment beam (B) can be performed on the basis of the fluoroscopic images.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing device comprising:
an image acquirer configured to acquire a plurality of fluoroscopic images by imaging a patient;
a trajectory generator configured to recognize a position of a part of interest shown in each of the plurality of fluoroscopic images and generate a trajectory of a state in which the part of interest has moved based on the recognized position of the part of interest; and
a selector configured to select a tracking method for tracking the part of interest based on the trajectory of the part of interest.

2. The medical image processing device according to claim 1, wherein the image acquirer acquires the fluoroscopic images by imaging the patient in a plurality of directions different from each other.

3. The medical image processing device according to claim 2, wherein the image acquirer acquires the plurality of fluoroscopic images that are continuously captured for a period of a length equivalent to at least one respiratory cycle of the patient.

4. The medical image processing device according to claim 3, wherein the trajectory generator recognizes the position of the part of interest according to template matching using a plurality of templates for recognizing the state of the part of interest.

5. The medical image processing device according to claim 4, wherein the trajectory generator generates the trajectory of the part of interest for each of the plurality of templates in each of a two-dimensional tracking process of tracking the part of interest in a two-dimensional coordinate system in the fluoroscopic image obtained by imaging the patient in one direction and a three-dimensional tracking process of tracking the part of interest in a three-dimensional coordinate system based on geometry information of an imaging device for simultaneously imaging the patient in a plurality of directions different from each other.

6. The medical image processing device according to claim 5, wherein the selector determines whether or not the part of interest is trackable by inputting the trajectory of the part of interest generated in each of the two-dimensional tracking process and the three-dimensional tracking process to a classifier and selects the tracking method based on the trajectory of the part of interest determined to be trackable.

7. The medical image processing device according to claim 6, further comprising a tracker configured to track the part of interest by the tracking method.

8. The medical image processing device according to claim 1, further comprising a likelihood calculator configured to calculate a likelihood of the part of interest shown in the fluoroscopic image,
wherein the selector selects the tracking method based on the trajectory of the part of interest and the likelihood of the part of interest.

9. The medical image processing device according to claim 8, wherein the part of interest is a marker placed inside the patient's body.

10. A treatment system comprising:
a medical image processing device including an image acquirer configured to acquire a plurality of fluoroscopic images by imaging a patient, a trajectory generator configured to recognize a position of a part of interest shown in each of the plurality of fluoroscopic images and generate a trajectory of a state in which the part of interest has moved based on the recognized position of the part of interest, and a selector configured to select a tracking method for tracking the part of interest based on the trajectory of the part of interest;
a display controller configured to cause a display device to display a display image indicating the trajectory of the part of interest;
an irradiator configured to irradiate a treatment target site of the patient indicated by the tracked part of interest with a treatment beam;
an irradiation controller configured to control the irradiation with the treatment beam; and
a patient table controller configured to move a position of a patient table on which the patient is fixed.

11. A medical image processing method comprising:
acquiring, by a computer, a plurality of fluoroscopic images by imaging a patient;
recognizing, by the computer, a position of a part of interest shown in each of the plurality of fluoroscopic images and generating a trajectory of a state in which the part of interest has moved based on the recognized position of the part of interest; and
selecting, by the computer, a tracking method for tracking the part of interest based on the trajectory of the part of interest.

12. A non-transitory computer-readable storage medium storing a program for causing a computer to:
acquire a plurality of fluoroscopic images by imaging a patient,
recognize a position of a part of interest shown in each of the plurality of fluoroscopic images and generate a trajectory of a state in which the part of interest has moved based on the recognized position of the part of interest, and
select a tracking method for tracking the part of interest based on the trajectory of the part of interest.
